(19) United States
(12) Patent Application Publication
(10) Pub. No.: US 2009/0010978 A1
(43) Pub. Date: Jan. 8, 2009

(54) Green tea jewelry

(75) Inventor: David Matthew Jensen, San Diego, CA (US)

Correspondence Address: MASTERMIND IP LAW PC, 421-A SANTA MARINA COURT, ESCONDIDO, CA, 92029, US

(73) Assignee: SoutherLee Incorporated, San Diego, CA (US)

(21) Appl. No.: 12/231,096

(22) Filed: Aug. 28, 2008

Related U.S. Application Data
(63) Continuation of application No. 11/379,556, filed on Apr. 20, 2006, now abandoned.

(51) Int. Cl. A61K 9/00; A61K 36/82; A61P 35/00; A61P 9/00

(52) U.S. Cl. 424/402; 424/729

(57) ABSTRACT

A method for making green tea jewelry is provided. The jewelry may be worn as a transdermal source of green tea components used as an alternative to or in addition to ingestion of green tea liquid or solids. Also provided is a kit for providing green tea jewelry in a substantially water resistant and air-tight package. The kit may comprise additional articles such as informational inserts and scents.

### BACKGROUND

The general health benefits derived from the consumption of green tea have been known for thousands of years. More recently, the use of green tea for general health benefits has become increasingly popular in more widespread geographic regions and by diverse cultures. Today, scientific evidence has linked certain positive health effects to various components of green tea. Specifically, positive effects in fending off cancer, heart disease, and other health benefits come from the green tea components of catechin polyphenols, polysaccharides, flavonoids, vitamin B complex, vitamin C, vitamin E, r-amino butyric acid, saponins, L-theanine, and fluoride. Polyphenols, otherwise known as catechins, have shown anti-inflammatory, anti-allergy, anti-viral, anti-microbial, anti-mutagenic, anti-carcinogenic, anti-aging, and anti-oxidative effects. Catechins found in green tea include gallocatechin, epicatechin (EC), epigallocatchin (EGC) and epigallocatechin gallate (EGCG).

Drinking green tea after conventional tea preparation, by steeping the tea plant or leaves in hot water, is the most common way the green tea and its beneficial components are introduced to the body. Other forms of ingestible green tea preparations exist, however, such as tablets and capsules, flavored beverages and “sports drinks,” ice cream, and dietary supplements such as candies, bars and chews. Some individuals are not able to or do not wish to ingest such products, for example, due to an aversion to the taste, diet concerns, or simply because of the mental stigma associated with consuming “pills.” Moreover, solid or crystalline forms of green tea require a significant amount of time for absorption by the body, and poor bioavailability often results in much of the dosage passing through the body unabsorbed. Other people would like sources of green tea components in addition to those already available. Therefore, there exists a need for alternate forms of delivering the beneficial effects of green tea and its components.

### SUMMARY OF THE INVENTION

This invention relates to transdermal delivery of green tea components. More specifically, this invention relates to wearable art, such as jewelry made from green tea. The jewelry may be worn in order to allow the skin of the subject to absorb the green tea components and enables the subject to enjoy the general health benefits of green tea without requiring its oral consumption.

### DETAILED DESCRIPTION

Throughout this specification, the terms “a” and “an” and variations thereof represent the phrase “at least one.” In all cases, the terms “comprising”, “comprises” and any variations thereof should not be interpreted as being limitative to the elements listed thereafter. Unless otherwise specified in the description, all words used herein carry their common meaning as understood by a person having ordinary skill in the art. In cases where examples are listed, it is to be understood that combinations of any of the alternative examples are also envisioned. The scope of the invention is not to be limited to the particular embodiments disclosed herein, which serve merely as examples representative of the limitations recited in the issued claims resulting from this application, and the equivalents of those limitations.

There are many different varieties of teas from the Camelia Sinensis plant, all of which are known as “green tea.” Examples of green tea include, but are not limited to Longjing, Hui Ming, Long Ding, Hua Ding, Qing Ding, Zhucha, Yu Lu, Xin Yang Mao Jian, Bi Luo Chun, Rain Flower, Yun Wu, Chun Mee, Da Fang, Huangshan Mao Feng, Lu An Guapian, Hou Kui, Tun Lu, Huo Qing, Hyson, Gyokuro, Matcha, Mecha, Sencha, Shincha, Genmaicha, Kabusecha, Kamairicha, Bancha, Hojicha, and Kukicha. Preferably, the tea is unfermented. Green teas may be processed in such a way as to be fashioned into wearable art, allowing for transdermal delivery of tea components that are beneficial to overall health. Such wearable art pieces should be designed such that a relatively large portion of the surface area comes into contact with the subject's skin for a prolonged period of time, such as for several hours, days or weeks.

Examples of such art pieces include, but are not limited to jewelry such as wrist bracelets, ankle bracelets, rings, necklaces, belly strings and the like. When the jewelry is secured to the body of a subject such that it fits snugly against the skin, the components of the tea used to fashion the jewelry may be absorbed into the skin and delivered to the body of the subject. Delivery of the green tea components by this type of vehicle enables the subject to obtain the positive effects of the green tea without being required to orally ingest it. The subject may enjoy a positive mental state when wearing such jewelry, due to the comfort of having a constant source of green tea available. Such positive mental state may, in itself, be beneficial to the subject's health.

Green tea may be processed for forming jewelry by any conventional method, readily apparent to any person having ordinary skill in the art. Such processes include, but are not limited to rolling and/or pressing leaves, which may then be made into strands. The processed plant may be formed directly into jewelry, for example, by braiding, weaving, knotting, tying or twisting.

In one embodiment of the invention, green tea is combined with a support used for forming such jewelry. The support is generally a piece of material that forms the general shape of the article. Appropriate supports should be capable not only of absorbing the green tea from the soaking liquid, but also capable of releasing it after it has dried, for delivery to the skin of a subject. Examples of such supports include, but are not limited to string, ribbon, rope, twine, jute, yarn, silk, cotton and cotton blend filaments, hemp, polyester filaments and the like. In a preferred embodiment, strands of hemp are employed as the support structure.

The green tea may be whole plant, plant pieces or powder, for example. Where the green tea employed is plant or plant pieces, it may be incorporated with the support by braiding, weaving, knotting, tying or twisting, for example. Methods of obtaining green tea powder are known by any person having skill in the art. For example, a conventional method comprises pulverizing green tea in a ball mill and screening with a sieve. Green tea powder may also be easily obtained from a variety of suppliers. Green tea powder may be added to liquid, such as water, and the support soaked in such liquid to absorb the green tea. The appropriate amount of green tea powder may vary depending on the type of tea and support used, the length and width of the jewelry, any additive ingredients affecting absorption and/or desorption, and the desired health effect. Appropriate amounts of green tea powder may vary widely and are considered to be easily determined by any person having ordinary skill in the art. For example, the ratio of green tea powder to support material may be in the range of about 0.25% to about 50% by weight, added to enough liquid to thoroughly and freely saturate the support material.

The support may be soaked in the green tea solution for a period of hours or days depending on the type of support used and the concentration of the green tea powder. Heating may reduce the amount of soaking time required for the support to absorb a desired amount of the green tea. The general variables involved in determining adequate soaking include time, temperature and concentration—an increase in any one or more of the variable tending to decrease the amount of soaking time required. Where optional dyes or other additives are included in the soaking solution, certain dyes and additives may not be amenable to high temperatures and soaking time may necessarily be prolonged. Supports should be free of textile sizing or other chemicals and may require pre-washing prior to soaking. Dry supports may be added directly to the green tea solution, or after having been pre-wet.

Optionally, dyes may be included in the green tea solution. Typical dyes include natural types comprising extracts and mineral mordants, and Procion® (Imperial Chemical Industries Ltd.) dyes. The amount of dye used is dependent upon the types of materials and dyes being employed, the temperature and time allowed for soaking, the concentration of the dye, and the desired result. Appropriate dye amounts can be easily determined by any person having ordinary skill in the art. Typical amounts are in the range of about 2 to 10 parts dye to each part green tea powder.

After soaking, the supports may be rinsed one or more times with liquid, such as water. This is especially important if optional dyes have been employed. The supports may be squeezed, pressed, rolled, wrung, twisted, folded, or brushed to remove excess liquid. The appropriate method can be easily determined based upon the type of support employed. After excess liquid has been removed, the supports may be dried by any appropriate method routinely used in the art. Appropriate methods include, but are not limited to hot or ambient air circulation, exposure to sunlight, “greenhouse” exposure, vacuum, and chemical treatment. Appropriate methods may be easily determined by any person having ordinary skill in the art.

After drying, the supports may be fashioned into strands which are then used to create the green tea jewelry. The strands may be any suitable length and width, taking into account the intended area where the finished product is to be worn, and any desired aesthetic considerations. Preferably, a plurality of stands is combined in an aesthetically pleasing design, for example, by braiding, weaving, knotting, tying or twisting.

Optional ingredients may be included in the jewelry, either as solids or by inclusion in the soaking liquid for absorption by the support. Examples of optional ingredients include, but are not limited to active (pharmaceutical) ingredients, adjuvants, emollients, scents, preservatives, and treatments to increase colorfast properties. Other optional ingredients include, but are not limited to vitamins and minerals, amino acids, other plant extracts, anti-oxidants such as grape seed, beta carotene, and co-enzyme Q10. Appropriate additives are readily apparent to any person having ordinary skill within the art.

Where color fixatives are desired, many commercial products are available depending on the type of support material and dye employed. Such fixatives may be added to the final rinse, applied after the final rinse and prior to drying, or applied after drying. In one embodiment, a quantity of vinegar is added to the final rinse water. The appropriate types of color fixatives and methods for applying them are easily determined by any person having ordinary skill in the art.

In one embodiment, castor oil is included in order to maintain the pliability of the finished product and to aid movement of the tea components into the subject's skin. Examples of other emollients include, but are not limited to glycerine, urea, olive oil, corn oil, cottonseed oil, sesame oil, wax, oleic acid, lanolin, cetyl alcohol, glyceryl monostearate, stearic acid, polyethylene glycol, polysorbate, shea butter, cocoa butter, paraffin, petrolatum, and the like. Emollients may be included during or after the last stages of rinsing or before the drying stage. In an alternate method, the emollient may be added after the drying stage.

The jewelry may optionally include aromatherapeutic scents. Examples of such scents include, but are not limited to peppermint, spearmint, jasmine, lavender, and naturally occurring or added green tea scent. The aromatherapeutic scent may be included within the body of the jewelry, or it may be separate, such as in an attached sachet. Optionally, scented oil may be applied once or periodically to the jewelry to maintain the scent for longer periods of time.

Optional scent oils may be added at the same time as the emollient, and where both are used, they may optionally be mixed together before being added. Appropriate amounts of emollients and scents vary widely depending upon the selected materials and the desired effect. For example, the ratio of green tea to castor oil might be in the range of about 2:1 to 5:1. The ratio of green tea to scent oil might be in the range of 15:1 to 45:1, for example. The foregoing ratios are not intended to be limiting, but rather to illustrate the relative amounts of the various ingredients that might be optionally employed. It should be understood that a wide variety of concentrations and results may be obtainable.

The jewelry may include a variety of decorative accents, being readily apparent to those having ordinary skill in the art. Such decorative accents include, but are not limited to ribbons, beads, bones, stones, gems, sequins, twigs, feathers, leaves, paper mache formations, pressed plants, charms and the like. The strands used to form the bracelet may optionally include color components, such as colored string, or the strands may optionally be dyed. The jewelry may include conventional clasps, fasteners, ball and loop mechanisms, other closure mechanisms, and the like. In a preferred embodiment, the ends of the jewelry are simply tied together, thus enabling the particular size and fit to be tailored to each individual subject. In a preferred embodiment, the entire jewelry piece is made from biodegradable materials.

The jewelry may be packaged in order to protect it during transport and to maintain the freshness of the product. Packages may include individual or multiple pieces of jewelry. The packages may be made of any appropriate material designed to repel moisture and remain relatively air-tight. Appropriate packaging materials are readily apparent to any person having ordinary skill in the art. Examples of appropriate materials include, but are not limited to plastic, cellophane, wax and wax composites and the like. Packages may include additional items, such as inserts serving as backing for the jewelry, and/or for providing informational and/or instructional material. Packages may also include additional scented items.

### EXAMPLE 1

### Preparing the Support (a)

Four parts natural dye and one part green tea powder are combined in a mixing vessel with enough water to cover the hemp support material to be treated. The material is in the form of strands. The hemp support material is pre-washed, and the damp hemp material is placed into the mixing vessel. The contents of the vessel are heated to approximately 180-200° F. for approximately 3 hours and allowed to cool to room temperature. The hemp material is rinsed thoroughly until no more color loss appears in the rinse water. A final rinse with a vinegar and water solution is performed. One part of castor oil is added for every 15 parts of dye, and one part of scent oil is added for every 30 parts of dye. Excess liquid is squeezed from the hemp material using a press. The material is dried under hot air circulation.

### EXAMPLE 2

### Making the Jewelry (a)

Several colors of hemp material are processed according to Example 1 by using different color dyes. Twelve stands of hemp material are twisted, knotted and woven into an aesthetically pleasing bracelet design. Natural coconut and wood beads are incorporated securely into the design. The finished product is about 28.6 cm in length and about 0.32 cm in width. Loose strands at each end of the bracelet serve as ties for securing the jewelry to the wrist of a subject. A sachet filled with scented material is tied to one end of the bracelet, inward from the loose strands used for tying the ends together. The sachet is comprised of paper Mache filled with scented pressed tea leaves.

### EXAMPLE 3

### Packaging the Jewelry (a)

A single bracelet is affixed to a cardboard backing with twist-ties. The cardboard backing contains product information and instructions, as well as anecdotal information. The cardboard and bracelet are inserted into a plastic pouch, designed to retain the scent for prolonged periods of time.

### EXAMPLE 4

### Preparing the Support (b)

One oz. of green tea powder is added to enough water to saturate and soak 1 pound of hemp strands. The dry hemp is placed in a mesh bag, submerged into the solution and allowed to soak for 3 days. 0.5 oz of castor oil is stirred into to the solution. The bag of hemp is then removed and excess water is wrung out. The hemp strands are removed from the bag and draped over lines to dry in the sun.

### EXAMPLE 5

### Making the Jewelry (b)

Six to 24 strands of the treated hemp are braided into an aesthetically pleasing necklace design. The finished design is about 34 cm in length and about 0.7 cm in width. An adjustable clasp is mechanism is incorporated to the end such that the overall size is adjustable about ±2 cm.

### EXAMPLE 6

### Packaging the Jewelry (b)

Three necklaces are placed into a cellophane package along with a plastic insert bearing information. A separate scent bag is also included. The cellophane package is sealed with adhesive, rendering it relatively water resistant and air-tight.

It should be understood that the foregoing examples are not intended to be limiting and are provided to illustrate just a few of the many embodiments of the invention. The broader spirit of the invention is readily apparent from the following claims.

1. Jewelry, the improvement being its capability of delivering at least one component of green tea to the skin of a subject, said jewelry comprising dried green tea solution.

2. (canceled)

3. The jewelry of claim 1, further defined as being a member selected from the group consisting of bracelet, necklace, ring, anklet, and belly chain.

4. The jewelry of claim 1, wherein said jewelry comprises green tea powder.

5. The jewelry of claim 1, wherein said jewelry further comprises a support.

6. The jewelry of claim 5, wherein said support comprises at least one material selected from the group consisting of string, ribbon, rope, twine, jute, yarn, silk, cotton, cotton blends, hemp, polyester and combinations thereof.

7. The jewelry of claim 5, wherein said support comprises green tea powder.

8. The jewelry of claim 1, further comprising dye.

9. The jewelry of claim 1, further comprising an emollient.

10. The jewelry of claim 9, wherein said emollient is at least one member selected from the group consisting of glycerin, urea, olive oil, corn oil, cottonseed oil, sesame oil, wax, oleic acid, lanolin, cetyl alcohol, glyceryl monostearate, stearic acid, polyethylene glycol, polysorbate, shea butter, cocoa butter, paraffin, petrolatum, castor oil and combinations thereof.

11. The jewelry of claim 1, further comprising scent.

12. The jewelry of claim 11, wherein said scent is at least one member selected from the group consisting of scent oil, pressed plant, sachet and combinations thereof.

13. (canceled)

14. The jewelry of claim 1, further comprising at least one decorative accent.

15. (canceled)

16. The jewelry of claim 1, further comprising a color fixative.

17. The jewelry of claim 1, further comprising at least one member selected from the group consisting of active ingredient, pharmaceutical, adjuvant, preservative, vitamin, mineral, amino acid, plant extract, anti-oxidant and combinations thereof.

18. A method for the transdermal delivery of at least one green tea component comprising wearing flush against the skin an article of jewelry comprising green tea.

19-21. (canceled)

22. A process for manufacturing jewelry comprising:
a) dissolving a quantity of green tea powder in an amount of water sufficient to cover support material to form a green tea solution;
b) soaking support material in the green tea solution;
c) rinsing the green tea solution from the support material;
d) drying the support material; and
e) fashioning the support material into jewelry.

23-39. (canceled)

40. A means for delivering at least one component of green tea to the skin of a subject comprising the wearing of jewelry comprising dried green tea solution.
